(19) United States
(12) Patent Application Publication
(10) Pub. No.: US 2019/0135720 A1
(43) Pub. Date: May 9, 2019

(54) METHOD OF PRODUCING ALCOHOL ALKOXYLATES

(71) Applicant: SHELL OIL COMPANY, Houston, TX (US)

(72) Inventors: Cornelius Mark BOLINGER, Sugar Land, TX (US); Heejae HUH, Houston, TX (US); Michael MIGUEZ, Houston, TX (US)

(21) Appl. No.: 16/241,135

(22) Filed: Jan. 7, 2019

(51) Int. Cl. C07C 41/03

(52) U.S. Cl. CPC C07C 41/03; C07C 43/135

(57) ABSTRACT

A method of producing alcohol alkoxylates comprising contacting a hydrocarbon mixture with a hydroformylation catalyst under hydroformylation conditions to form a product mixture comprising one or more alcohols and formate esters; treating the product mixture to reduce the concentration of formate esters to produce an alcohol stream; and contacting the alcohol stream and an epoxide with a potassium hydroxide catalyst under alkoxylation conditions to produce one or more alcohol alkoxylates.

### FIELD OF THE INVENTION

The invention relates to a method of producing alcohol alkoxylates.

### BACKGROUND

A large variety of products useful, for instance, as nonionic surfactants, wetting and emulsifying agents, solvent, and chemical intermediates, are prepared by the alkoxylation reaction of alkylene oxides with alcohols. Such ethoxylates, and to a lesser extent corresponding propoxylates and compounds containing mixed oxyethylene and oxypropylene groups, are widely employed as nonionic detergent components of commercial cleaning formulations for use in industry and in the home.

### SUMMARY OF THE INVENTION

The invention provides a method of producing alcohol alkoxylates comprising contacting a hydrocarbon mixture with a hydroformylation catalyst under hydroformylation conditions to form a product mixture comprising one or more alcohols and formate esters; treating the product mixture to reduce the concentration of formate esters to produce an alcohol stream; and contacting the alcohol stream and an epoxide with a potassium hydroxide catalyst under alkoxylation conditions to produce one or more alcohol alkoxylates.

### DETAILED DESCRIPTION

The invention provides a method of producing alcohol alkoxylates where the amount of byproducts, including carbon monoxide and/or polyalkyleneglycols, is reduced. It is believed that the amount of byproducts is reduced by reducing the amount of formate esters in the alcohol feed to the alkoxylation step. The invention includes a treatment or other step to reduce the amount of formate esters in the alcohol stream. The treatment step can include hydrogenation or saponification.

### Hydroformylation

The one or more alcohols that are alkoxylated may be produced in any way. In a preferred embodiment, the alcohols are produced by hydroformylation of one or more olefins. Hydroformylation comprises the reaction of olefinic hydrocarbon compounds with carbon monoxide and hydrogen in the presence of a catalyst. For example, alcohols may be prepared by any of the methods described in U.S. Pat. Nos. 3,239,571; 3,274,263; 3,420,898; 5,849,960; 6,150,222; and 6,222,077 all of which are herein incorporated by reference.

### Feed

The feed to the hydroformylation process comprises one or more hydrocarbons, preferably having from 4 to 20 carbon atoms, and more preferably from 8 to 18 carbon atoms. The hydrocarbon is preferably an olefinic compound, and it may be an alpha-olefin or an internal olefin. If a single hydrocarbon is used then a single alcohol will be produced, but in some embodiments, a mixture of hydrocarbons are used (i.e., having varying carbon chain lengths) which result in a mixture of alcohols

The reaction may be carried out in the presence of a solvent. The solvent should be inert in that it does not substantially interfere with the hydroformylation reaction. Suitable solvents include alkanes, alcohols and ethers.

The ratio of hydrogen to carbon monoxide fed to the reaction may be varied significantly. For example, the ratio of hydrogen to carbon monoxide may be within the range of from 1:1 to 10:1. The ratio has an effect on the product slate produced because aldehydes are formed in the reaction of 1 mole of hydrogen and 1 mole of carbon monoxide with the hydrocarbon while alcohols are formed in the reaction of 2 moles of hydrogen and 1 mole of carbon monoxide with the hydrocarbon.

### Hydroformylation Catalyst

The hydroformylation catalyst may be any suitable catalyst known in the art. The catalyst may comprise a metal-ligand complex. The catalyst may comprise cobalt, rhenium, osmium, iridium or platinum. The catalyst may also comprise a phosphorous containing ligand. The ligand may comprise a tertiary organo phosphorous compound in which the phosphorous is trivalent (also referred to as phosphines), for example tributyl phosphine or triphenyl phosphine. The catalyst may comprise a complex of a metal with carbon monoxide and a tertiary organophosphine.

The concentration of catalyst is not critical, but in general, a higher concentration of catalyst provides a faster reaction rate. Ratios of catalyst to hydrocarbon in the range of from 1:1000 to 10:1 are normally satisfactory.

### Hydroformylation Conditions

The hydroformylation reaction is preferably carried out in the liquid phase where the olefinic compound is intimately contacted with the carbon monoxide and hydrogen in the presence of the hydroformylation catalyst. The reaction temperature may be in the range of from about 100 to about 300° C. and preferably from about 150 to about 210° C. The reaction pressure may be in the range of from atmospheric to about 14 MPa.

### Product Mixture

The hydroformylation reaction predominantly produces aldehydes and alcohols. As described above, the hydroformylation conditions can be adjusted to adjust the respective amounts of alcohols and aldehydes produced. The products have one more carbon atom than the corresponding hydrocarbon feed as a result of the reaction. The products can be subjected to suitable catalyst and product separation steps, for example, solvent extraction, distillation, fractionation, or adsorption. Catalyst, unreacted feeds and solvent may be recycled back to the hydroformylation reaction step.

Various commercial alcohol products are produced in this or similar processes including NEODOL™ 67, which includes a mixture of C₁₆ and C₁₇ alcohols of the formula R—OH, wherein R is a branched alkyl group having a branching index of about 1.3, sold by Shell Chemical LP. NEODOL™ as used throughout this text is a trademark. Shell Chemical LP also manufactures a C₁₂/C₁₃ analogue alcohol of NEODOL™ 67, which includes a mixture of C₁₂ and C₁₃ alcohols of the formula R—OH, wherein R is a branched alkyl group having a branching index of about 1.3. Another suitable example is EXXAL™ 13 tridecylalcohol (TDA), sold by ExxonMobil, which is of the formula R—OH wherein R is a branched alkyl group having a branching index of about 2.9 and having a carbon number distribution wherein 30 wt. % is C₁₂, 65 wt. % is C₁₃ and 5 wt. % is C₁₄. Yet another suitable example is MARLIPAL® tridecylalcohol (TDA), sold by Sasol, which product is of the formula R—OH wherein R is a branched alkyl group having a branching index of about 2.2 and having 13 carbon atoms.

In addition to the desired alcohol and aldehyde products described above, the process also produces formate esters. The formate esters are formed by the reaction of aldehydes with CO and H₂. The formate ester concentration in the hydroformylation product can be measured by determining the cold saponification number using known techniques, for example ASTM D94 or DIN 51559.

### Reduction of Formate Esters

### Saponification

The formate esters in the product stream can be reduced by reaction with an aqueous base, for example, sodium hydroxide or potassium hydroxide to hydrolyze the formate ester. The reaction produces a carboxylate salt, for example sodium formate, and an alcohol. This reaction is referred to as a saponification reaction. In one embodiment, the preferred aqueous base is sodium hydroxide.

The saponification reaction can be carried out under any suitable saponification conditions known to one of ordinary skill in the art. For example, the saponification may be carried out at a temperature in the range of from 40 to 100° C. A more concentrated solution of the aqueous base is preferably used to limit the amount of water that is added to the product that will need to be removed.

### Hydrogenation

The product stream comprising alcohols, aldehydes and formate esters may be subjected to a hydrogenation step to reduce the concentration of formate esters. The hydrogenation step may involve the use of a heterogeneous catalyst, and optionally the addition of water. In the hydrogenation step, the content of formate esters is reduced by conversion of the formate esters to an alcohol and carbon monoxide. In one example, a sulfided bimetallic catalyst can be used in the hydrogenation step to convert formate esters to low levels while being resistant to poisoning by the formic acid and potential sulfur impurities in the feed to the hydrogenation step. In another embodiment, the formate esters can be hydrolyzed with water to form the alcohol and formic acid.

### Controlling Hydroformylation Conditions

The reaction conditions in the hydroformylation reaction may be controlled to minimize the amount of formate esters formed. For example, the temperature of the reaction, the catalyst or ligand concentration, and/or the hydrogen and carbon monoxide composition and pressure may be controlled to affect the amount of formate esters formed.

In one embodiment, water can be injected into the hydroformylation reactor to reduce the amount of formate esters formed. If water is injected it is important to control the amount to prevent the formation of a free water phase in the reactor. Further, it is important to ensure good mixing of the water with the feed to the hydroformylation reaction.

In one embodiment, any combination of controlling the hydroformylation reaction conditions, hydrogenation and saponification can be employed to reduce the concentration of formate esters in the product stream.

### Alkoxylation

The alcohol may be alkoxylated by reacting with alkylene oxide in the presence of an appropriate alkoxylation catalyst to form an alcohol alkoxylate. The alkoxylation step serves to introduce a desired average number of alkylene oxide units per mole of alcohol alkoxylate, wherein different numbers of alkylene oxide units are distributed over the alcohol alkoxylate molecules. For example, treatment of an alcohol with 7 moles of alkylene oxide per mole of alcohol results in the alkoxylation of each alcohol molecule with an average of 7 alkylene oxide groups, although a substantial proportion of the alcohol will be combined with more than 7 alkylene oxide groups and an approximately equal proportion will be combined with less than 7. In a typical alkoxylation product mixture, there may also be a minor proportion of unreacted alcohol.

### Feed

The alcohol that is fed to the alkoxylation step is the alcohol produced by the above described hydroformylation reaction. The alcohol has from 5 to 21 carbon atoms and preferably from 9 to 19 carbon atoms. The alcohol may be a straight chain or branched alcohol. The branching may occur at the 2-position and may comprise methyl, ethyl, propyl, butyl, pentyl or larger hydrocarbyl moieties.

### Catalyst

The alkoxylation catalyst may be a strong base, preferably comprising an alkali metal or an alkaline earth metal. The catalyst may comprise lithium, sodium, potassium, cesium. Alternatively, the catalyst may comprise magnesium, calcium or barium. The catalyst may be a metal hydroxide, for example potassium hydroxide or sodium hydroxide which are commonly used commercially. Alternatively, a double metal cyanide catalyst may be used, as described in U.S. Pat. No. 6,977,236. Still further, a lanthanum-based or a rare-earth metal-based alkoxylation catalyst may be used, as described in U.S. Pat. Nos. 5,059,719 and 5,057,627.

### Alkoxylation Conditions

The alkoxylation reaction temperature may range from 90° C. to 250° C., suitably 120 to 220° C., and super atmospheric pressures may be used if it is desired to maintain the alcohol substantially in the liquid state. The amount of alkoxylation catalyst in the reaction is of from 0.01 to 5 wt. %, more suitably 0.05 to 1 wt. %, most suitably 0.1 to 0.5 wt. %, based on the total weight of the catalyst, alcohol and alkylene oxide (i.e. the total weight of the final reaction mixture). In one embodiment, a Lewis base is present in the alkoxylation reaction.

### Product

The alkoxylation produces alcohol alkoxylates. In addition, any formate esters that are fed to the alkoxylation reaction are converted into carbon monoxide and alkylene glycols. This reaction is illustrated in U.S. Pat. No. 4,474,744 that describes a process for decarbonylating an alkyl formate to form carbon monoxide and an alcohol which reaction is catalyzed by Lewis bases in the presence of an epoxide. The formation of carbon monoxide can thus be reduced by reducing the formate ester content in the alcohol fed to the alkoxylation reaction. The alkylene glycols formed from the converted formate esters can react further with alkylene oxide, to form polyalkylene glycols, for example, polyethylene glycol.

The invention provides a process to reduce the amount of carbon monoxide and polyalkylene glycols formed in the alkoxylation reaction by reducing the concentration of formate esters in the alcohol fed to the alkoxylation reaction.

1. A method of producing alcohol alkoxylates comprising:
a. contacting a hydrocarbon mixture with a hydroformylation catalyst under hydroformylation conditions to form a product mixture comprising one or more alcohols and formate esters;
b. treating the product mixture to reduce the concentration of formate esters to produce an alcohol stream;
c. contacting the alcohol stream and an epoxide with a potassium hydroxide catalyst under alkoxylation conditions to produce one or more alcohol alkoxylates.

2. The method of claim 1 wherein the hydrocarbon mixture comprises hydrocarbons having from 4 to 20 carbon atoms.

3. The method of any of claims 1-2 wherein the hydroformylation conditions of step a) are controlled to produce less formate esters.

4. The method of claim 3 further comprising controlling the temperature, catalyst or ligand concentration, or hydrogen and carbon monoxide composition and pressure to reduce the production of formate esters.

5. The method of any of claims 1-4 wherein step b) comprises hydrogenating the product mixture to reduce the concentration of formate esters.

6. The method of any of claims 1-5 wherein step b) comprises contacting the product mixture with an aqueous base to convert at least a portion of the formate esters into alcohols and formates.

7. The method of claim 6 wherein the aqueous base comprises sodium hydroxide, lithium hydroxide, potassium hydroxide and cesium hydroxide.

8. The method of any of claims 1-7 wherein the epoxide is selected from the group consisting of ethylene oxide, propylene oxide, butylene oxide and mixtures thereof.

9. The method of any of claims 1-8 wherein a Lewis base is also present in step c).

10. The method of any of claims 1-8 wherein carbon monoxide is also formed in step c).

11. The method of any of claims 1-9 wherein one or more polyalkeneglycols are also formed in step c).
